(19) United States
(12) Patent Application Publication
(10) Pub. No.: US 2013/0012984 A1
(43) Pub. Date: Jan. 10, 2013

(54) SURGICAL INSTRUMENT FOR GRASPING AN ELONGATED MEMBER

(75) Inventor: Daniel Paxton Wall, Medina, TN (US)

(73) Assignee: Warsaw Orthopedic, Inc., Warsaw, IN (US)

(21) Appl. No.: 13/178,008

(22) Filed: Jul. 7, 2011

(51) Int. Cl. A61B 17/28

(52) U.S. Cl. 606/206; 606/205

(57) ABSTRACT

A surgical instrument for grasping an elongated member. The surgical instrument may include a handle and a hollow first shaft with a first end attached to the handle and an opposing second end. A second shaft may be positioned in a channel of the first shaft. The second shaft may include a proximal end attached to the handle and an opposing distal end. A gripping member may extend across the channel and is attached to the distal end of the second shaft. The gripping member has a pair of arms positioned on opposing sides of the channel that flare outward away from the distal end of the second shaft and terminate at tips. The gripping member is movable along the channel between a first position with the tips a first distance apart and a second position with the tips a smaller second distance apart.

### BACKGROUND

The present application is directed to a device for grasping an elongated member during a surgical procedure and, more particularly, to a device with a gripping mechanism for securely gripping and releasing the elongated member.

Elongated members, such as rods and cables, are used in a variety of surgical procedures. One example includes spinal rods used in the treatment of spinal disorders such as degenerative disc disease, disc herniations, scoliosis or other curvature abnormalities, and fractures. Another example includes a rod for the treatment of a fractured long bone such as the femur, tibia, fibula, or humerus. In the various embodiments, the elongated member is attached to one or more bones with anchors. The elongated member includes a length to span across the treatment area within the patient. The elongated member may inhibit relative motion between the bones, such as to inhibit movement between sections of a fractured long bone or between adjacent vertebral bodies during a spinal-fusion surgery. The elongated member may also have a dynamic structure to preserve motion between adjacent bones, such as to allow movement between adjacent vertebral bodies.

Various instruments are used by doctors for insertion and placement of an elongated member within the patient. The instruments generally have an elongated shape with a handle at a first end and a gripping section at an opposing second end. The handle is operatively connected to the gripping section to move the gripping section between open and closed orientations. In use, the doctor grasps the handle and manipulates the gripping section to receive and hold the elongated member. The doctor than uses the tool to place the elongated member into the patient. The handle is further manipulated to move the gripping section to the open orientation to remove the tool from the elongated member and from the patient.

Existing tools suffer from a variety of issues. The tool should be configured for the gripping section to securely grasp the elongated member for insertion and placement within the patient. The tool should further be configured for the gripping section to detach from the elongated member without displacing the positioning of the elongated member within the patient.

### SUMMARY

The present application is directed to surgical instruments for grasping an elongated member. One surgical instrument includes a handle and a first shaft with a first end attached to the handle and an opposing second end with the first shaft being hollow with an interior channel. A second shaft is positioned in the channel of the first shaft and includes a proximal end attached to the handle and an opposing distal end. A gripping member extends across the channel and is attached to the distal end of the second shaft. The gripping member has a pair of arms positioned on opposing sides of the channel that flare outward away from the distal end of the second shaft and terminate at tips. The gripping member is movable along the channel between a first position with the tips a first distance apart and a second position with the tips a smaller second distance apart. The gripping member extends across the channel in both the first and second positions. The gripping member is also spaced away from the handle by the second shaft.

Another surgical instrument includes a handle and a hollow first shaft with a first end attached to the handle and an opposing second end. The first shaft has a channel with a proximal section having a first width and a distal section having a larger second width that tapers outward towards the second end. A second shaft is positioned in the channel. A gripping member is attached to the second shaft and is positioned in the distal section of the channel and away from the handle. The gripping member includes a base that extends across the distal section of the channel and a pair of arms extending outward from the base and positioned on opposing sides of the distal section of the channel. Each of the arms has a tip opposite from the base. The gripping member includes a tapered width that is narrower at the base and wider at the tips. The base is narrower than the distal section of the channel and the tips are wider than the distal section of the channel. The gripping member is movable along the distal section of the channel between a first position with the tips of the arms positioned a first distance apart and a second position with the tips in closer proximity to the handle and the tips of the arms positioned a smaller second distance apart.

Methods of grasping an elongated member using a surgical instrument during a surgical procedure are also disclosed. One method includes positioning a pair of arms on a gripping member in an open orientation with distal sections of the arms spaced apart a first distance. While in the open orientation, positioning the pair of arms on opposing sides of the elongated member. The method includes moving proximal sections of the pair of arms into a tapered section of a tube and contacting outer surfaces of the arms against tapered ramps. The method includes moving the proximal sections along the tapered ramps and forcing the arms closer together and compressing the distal sections of arms against the elongated member. The method includes placing the gripping member in a closed orientation secured to the elongated member and maintaining the arms in an enlarged distal section of the tapered section of the tube and in contact with the tapered ramps.

The various aspects of the various embodiments may be used alone or in any combination, as is desired.

### BRIEF DESCRIPTION OF THE DRAWINGS

FIG. 1 is a perspective view of an instrument in a closed orientation grasping an elongated member.

FIG. 2 is a perspective view of an instrument in an open orientation.

FIG. 3 is a partial perspective view of shaft.

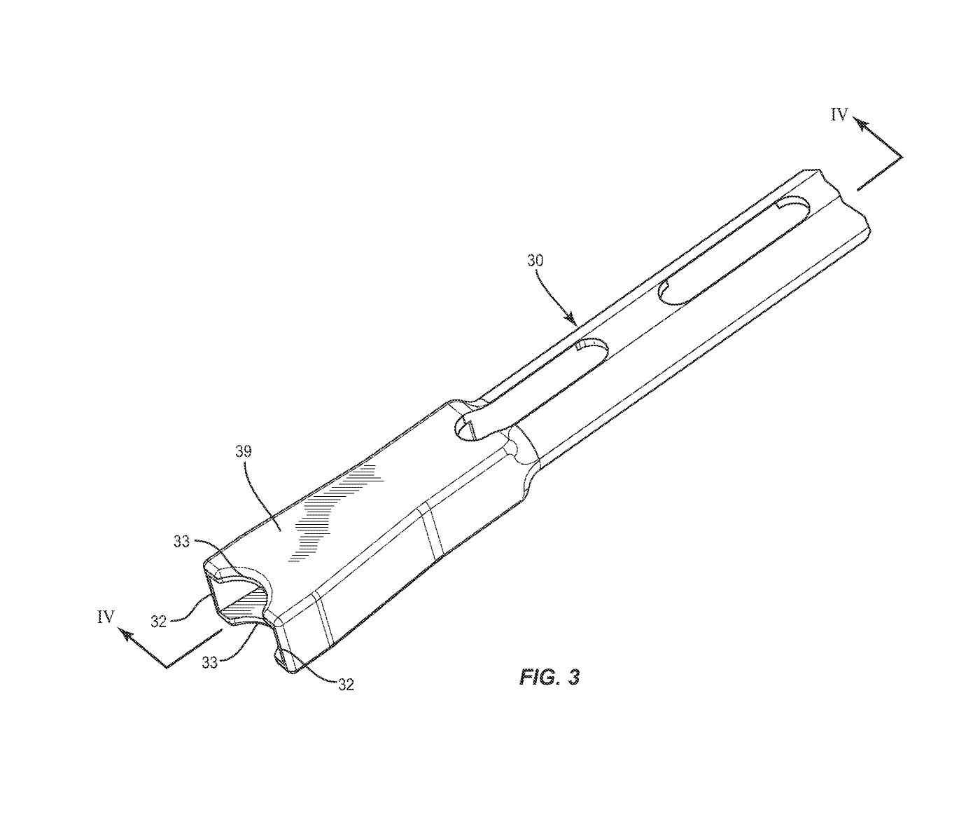

FIG. 4 is a sectional view of the shaft cut along line IV-IV of FIG. 3.

FIG. 5 is a perspective view of a second shaft and a gripping member.

FIG. 6 is a partial perspective view of a second shaft and a gripping member.

FIG. 7 is a partial perspective view of a second shaft and a gripping member.

### DETAILED DESCRIPTION

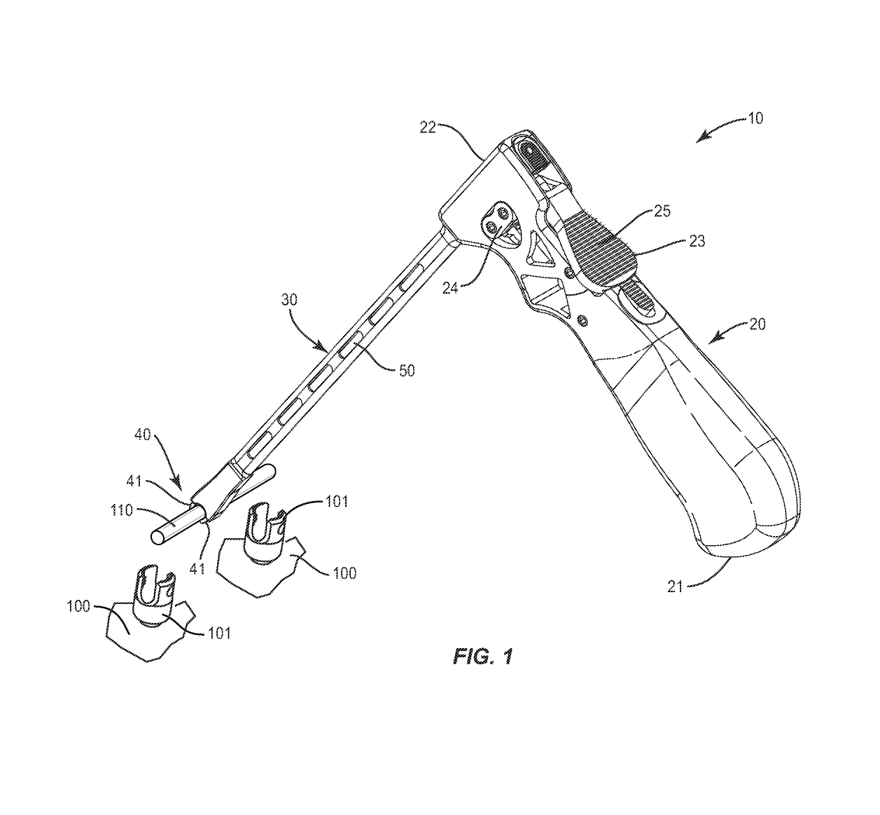

The present application is directed to a surgical instrument for grasping an elongated member. As illustrated in FIG. 1, the instrument 10 generally includes a handle 20, first shaft 30, second shaft 50, and gripping member 40. The gripping member 40 is configured to move relative to the shaft 30 and move from an open orientation to a closed orientation to grasp the elongated member 110. The instrument provides for grasping and manipulating the elongated member 110. Once finished, the instrument 10 is configured to be returned to the open orientation to release the elongated member 110.

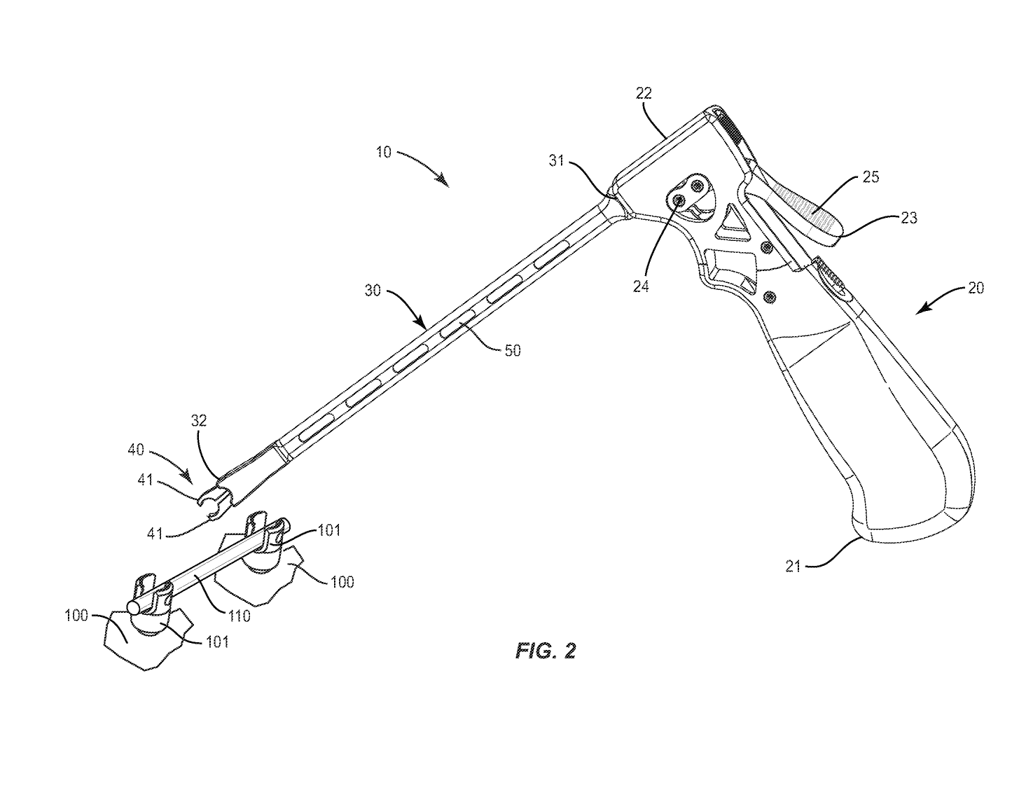

The instrument 10 is adjustable between the closed and open orientations. In the closed orientation as illustrated in FIG. 1, opposing arms 41 on the gripping member 40 are forced together to grasp the elongated member 110. The instrument 10 can then be manipulated to place the elongated member 110 into anchors 101 that are attached to bones 100. Once the elongated member 110 is placed in the patient, the instrument 10 is moved to the open orientation as illustrated in FIG. 2. The gripping member 40 is moved relative to the shaft 30 causing the arms 41 to move apart to release the elongated member 110. The positioning of opposing arms 41 on the sides of the elongated member 110 allows for the instrument 10 to be moved directly away from the elongated member 110 when releasing the elongated member 110.

The handle 20 is shaped to be grasped by the doctor to manipulate the instrument 10 and also to control and orientation of the gripping member 40. As illustrated in FIGS. 1 and 2, the handle 20 includes a body 21 that is grasped by the doctor. The body 21 may be ergonomically shaped and/or include a surface features such as a knurled finish, grooves, bumps, etc. to facilitate grasping by the doctor.

The handle 20 further includes a head 22 at the end of the body 21. The head 22 includes a lever 23 attached at a pivot 24. The lever 23 includes an elongated shape with a contact section 25 positioned on a first side of the pivot 24, and a receptacle (not illustrated) positioned in an interior of the head 22 and on an opposing second side of the pivot 24.

The first shaft 30 extends outward from the head 22. The shaft 30 includes an elongated shape with a first end 31 at the head 22 and an opposing second end 32. The shaft 30 includes an enlarged section 39 with an increased width at the second end 32. Scallops 33 may be cut into opposing sides of the second end 32 to receive the elongated member 110. The shaft 30 may have a variety of cross-sectional shapes, including but not limited to circular, oval, and polygonal.

The shaft 30 includes a hollow interior channel 34 to receive the second shaft 50 and the gripping member 40. The channel 34 includes a width w measured between opposing sides. The width w of the interior channel 34 is substantially constant through the majority of the shaft 30 from the first end 30 to the enlarged section 39. This narrower section of the channel 34 is positioned on a proximal side of the enlarged section 39.

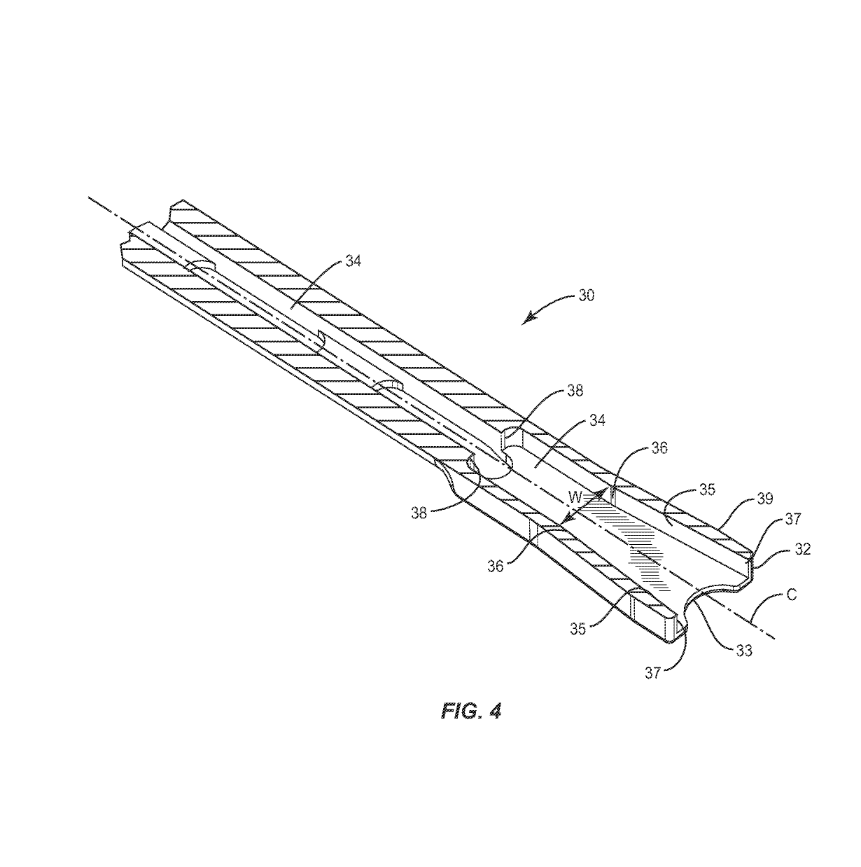

The width w increases in the enlarged section 39 as illustrated in FIG. 4. The enlarged section 39 includes outwardly-angled ramps 35 that extend away from the centerline C on opposing sides of the channel 34. The ramps 35 cause the width w of the channel 34 to increase towards the second end 32. Each of the ramps 35 may be positioned at a common angle relative to a centerline C of the channel 34. Alternatively, the ramps 35 may be positioned at different angles. In one embodiment, the channel 34 includes a single outwardly-angled ramp 35 with the opposing side being parallel to the centerline C.

The ramps 35 extend from a first point 36 to a second point 37. The length of the ramps measured between the points 36, 37 may vary. In one embodiment, the ramps 35 extend along an entirety of the enlarged section 39. In the embodiment shown in FIG. 4, the ramps 35 extend along a limited length of the enlarged section 39 with a portion of the enlarged section 39 have a substantially constant width w. As illustrated in FIG. 4, the ramps 35 may extend to the second end 32 (i.e., the point 37 is at the second end 32), or may be spaced inward from the second end 32. The opposing ramps 35 may have the same or different lengths, and may be positioned at the same or different longitudinal positions along the channel 34.

Ledges 38 are positioned inward from the ramps 35 to transition the channel 34 from the reduced width of the proximal section that extends through the majority of the shaft 30 to the larger width at the enlarged section 39. The ledges 38 extend transverse to the centerline C of the channel 34. In one embodiment, the ledges 38 are perpendicular to the centerline C.

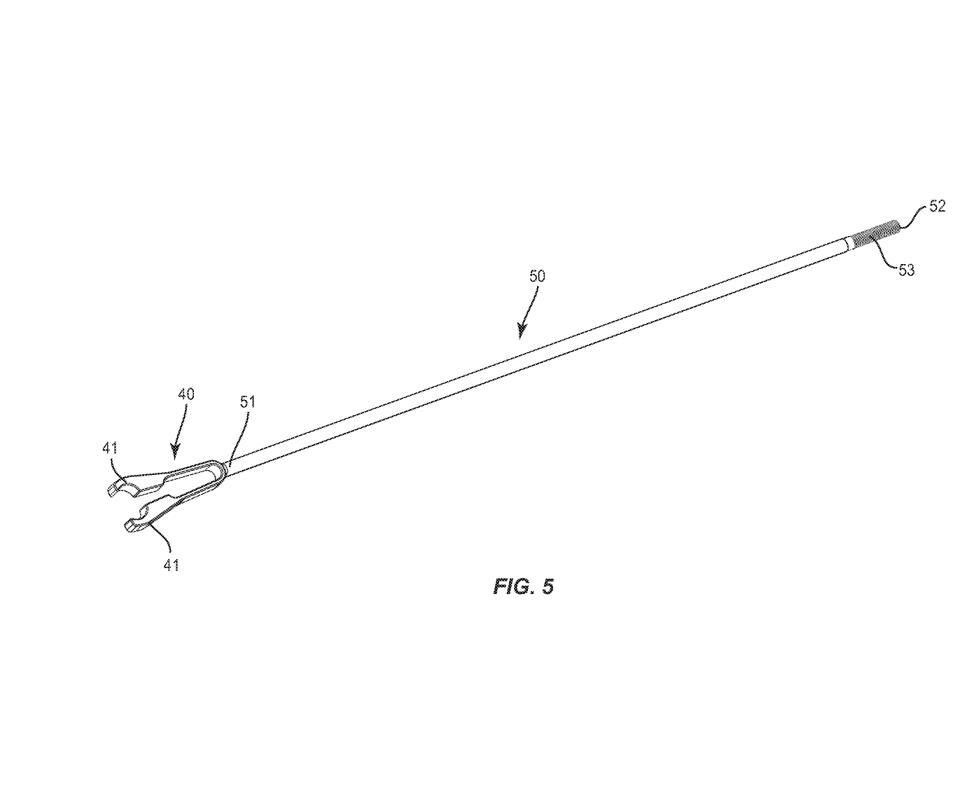

The second shaft 50 extends along the channel 34 of the first shaft 30. As illustrated in FIG. 5, the second shaft 50 includes a first end 51 that is attached to the gripping member 40. The second shaft 50 also includes a second end 52 that is attached to the lever 23. The second end 52 may include threads 53 for attaching with the lever 23. The second shaft 50 is narrower than the channel 34, therefore, pivoting movement of the lever 23 causes longitudinal movement of the second shaft 50 relative to the first shaft 30. In one embodiment, the second shaft 50 is solid.

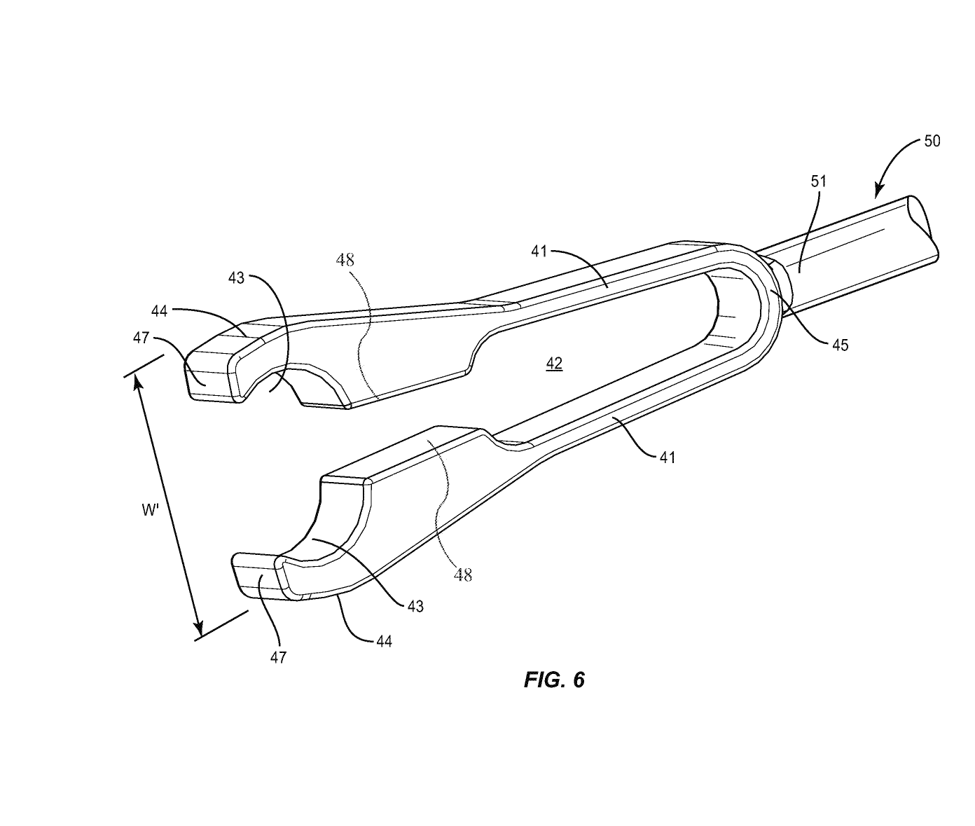

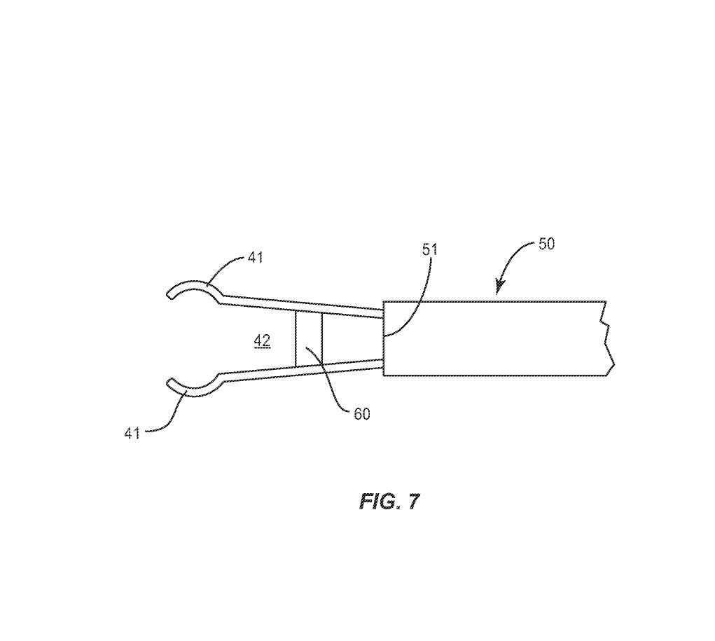

The gripping member 40 is attached to the first end 51 of the second shaft 50 and is spaced away from the handle 20 by the second shaft 50. The gripping member 40 includes a pair of opposing arms 41 that are spaced apart by an interior space 42. As illustrated in FIG. 6, the arms 41 may be attached together at a base 45 with the gripping member 40 formed as a single monolithic piece. The base 45 may be attached to the first end 51 in a variety of manners, including but not limited to a mechanical fastener such as a screw or rivet, by a soldering or welding structure, or by an adhesive. In one embodiment as illustrated in FIG. 6, the gripping member 40 includes a U-shape with the base 45 forming a closed end and an opposing end formed between the arms 41 being open. The gripping member 40 may further be structured with the arms 41 being separate and each individually attached to the first end 51 of the second shaft 50 as illustrated in FIG. 7. The arms 41 may be attached to the second shaft 50 in a similar manner as described above. In one embodiment, the first end 51 of the shaft 50 includes cavities that receive the ends of the arms 41.

A biasing member 60, such as a spring or elastic material, may be positioned in the interior space 42. The biasing member 60 contacts against and biases the arms 41 away from each other. The biasing member 60 may include a variety of configurations, including but not limited to a spring, such as a coil spring, or an elastic material.

The gripping member 40 is constructed such that the arms 41 are biased apart and may be compressed inward towards each other. Further, the gripping member 40 extends across the interior channel 34 of the first shaft 30. The portion of the gripping member 40 extending across the channel 34 may include one or more of the base 45, one or both arms 41, and the biasing member 60.

Each of the arms 41 includes a receptacle 43 at or in proximity to a distal end 47. The receptacles 43 together form a holding section that receives the elongated member 110 when the instrument 10 is in the closed orientation. The shape of the receptacles 43 may match the exterior shape of the elongated member 110 to facilitate a secure grasp on the elongated member 110. Just inward from the receptacles 43 is a relatively narrow neck formed between surfaces 48. The narrow neck blocks any further movement of the elongated member 110 proximally within the interior space 42 and maintains the elongated member 110 aligned in the receptacles 43. The surfaces 48 may be planar as illustrated in FIG. 6.

The arms 41 further include an outer surface 44 that faces away from the interior space 42. Sections of the outer surface 44 contact against the sides of the channel 34 during longitudinal movement of the gripping member 40 as will be explained in more detail below.

The gripping member 40 includes a width w′ measured between the outer surfaces 44. The width w′ various along the length of the gripping member 40 with an enlarged width w′ at the receptacles 43 and a reduced width w′ at the proximal end of the arms 41. The enlarged width w′ at the receptacles 43 is greater than the width w of the channel 34 to force the arms 41 inward when the gripping member 40 moves proximally along the channel 34.

In use, the doctor grasps the body 21 of the handle 20 to hold the instrument 10. The instrument 10 is in the open orientation with the distal ends 46 of the arms 41 extending outward beyond the second end 32 of the first shaft 30. Further, the arms 41 are spaced apart an amount to extend over the elongated member 110. The doctor then moves the instrument 10 to position the elongated member 110 within the interior space 42 between the arms 41. The doctor moves the instrument 10 and aligns the elongated member 110 with the receptacles 43 on the inner surfaces of the arms 41.

Once aligned, the doctor contacts the contact section 25 and moves the lever 23 about the pivot 24. This pivoting movement causes the second shaft 50 and the gripping member 40 to longitudinally move in a proximal direction along the channel 34 in the first shaft 30. As the gripping member 40 moves proximally along the channel 34, the outer surfaces 44 of the arms 41 contact against the surfaces of the ramps 35. Continued movement of the gripping member 40 into the narrower channel 34 causes the arms 41 to further move together. This movement causes the arms 41 move contact against opposing sides of the elongated member 110. The inward movement of the gripping member 40 may also cause the elongated member 110 to seat into the scallops 33 at the second end 32 of the first shaft 30. The distal ends 47 or the arms 41 remain outward beyond the end 32 of the shaft 32.

A locking mechanism may secure the instrument 10 in the closed orientation. The locking mechanism may include a member that extends between the lever 23 and the body 21 to maintain the position of the lever 23. Another locking mechanism includes a fastener that extends through the first shaft 30 and into the channel 34 to contact against and maintain the longitudinal position of the second shaft 50. In another embodiment, the instrument 10 does not include a locking feature and the doctor maintains the position of the lever 23 to keep the instrument 10 in the closed orientation.

In the closed orientation, the gripping member 40 is positioned in the enlarged section 39. In one embodiment, the entire gripping member 40 is positioned in the enlarged section 39 and away from the narrower section shaft 30 (i.e., the gripping member 40 is positioned distally relative to the ledges 38). In the closed orientation, the outer surfaces 44 of the arms 41 are in contact with the ramps 35 of the channel 34.

In the closed orientation, the doctor positions the elongated member 110 within the patient using the handle 20. In one method, the doctor positions the elongated member 110 within the anchors 101 that extend from the bones 100. Once the elongated member 110 is positioned in the patient, the doctor releases the elongated member 110 from the instrument 10. This includes releasing the locking mechanism or otherwise causing the second shaft 50 and gripping member 40 to move distally along the channel 34. This distal movement causes the gripping member 40 to move outward away from the narrower sections of the channel 34 towards wider sections. The arms 41 follow along the surfaces of the channel 34 and move away from one another thus causing the receptacles to release from the elongated member 110.

Because the arms 41 are positioned on opposing sides of the elongated member 110, the instrument 10 can be moved directly away from the elongated member 110 once it is released. There is no need to swivel or otherwise pivot the instrument 10 relative to the elongated member 110 to release the arms 41.

The instrument 10 may be used during surgical procedures on living patients. The instrument 10 may also be used in a non-living situation, such as within a cadaver, model, and the like. The non-living situation may be for one or more of testing, training, and demonstration purposes.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A surgical instrument for grasping an elongated member comprising:
a handle;
a first shaft with a first end attached to the handle and an opposing second end, the first shaft being hollow with an interior channel;
a second shaft positioned in the channel of the first shaft and including a proximal end attached to the handle and an opposing distal end; and
a gripping member extending across the channel and attached to the distal end of the second shaft, the gripping member having a pair of arms positioned on opposing sides of the channel that flare outward away from the distal end of the second shaft and terminate at tips;
the gripping member movable along the channel between a first position with the tips a first distance apart and a second position with the tips a smaller second distance apart, with the gripping member extending across the channel in both the first and second positions;
the gripping member being spaced away from the handle by the second shaft.

2. The surgical instrument of claim 1, wherein the gripping member is a monolithic single piece.

3. The surgical instrument of claim 2, wherein the gripping member further includes a base with the pair of arms extending outward from the base, the base being attached to the distal end of the second shaft and extending across the channel.

4. The surgical instrument of claim 3, wherein the gripping member includes a closed end at the base and an open end between the pair of arms, the closed end being narrower than the open end.

5. The surgical instrument of claim 1, wherein the channel of the first shaft includes a tapered section formed by a pair of opposing ramps that angle outward away from a centerline of the channel.

6. The surgical instrument of claim 5, wherein the ramps terminate at the second end of the first shaft.

7. The surgical instrument of claim 1, further comprising a biasing member positioned between the arms to bias the arms apart, the biasing member extending across the channel.

8. A surgical instrument for grasping an elongated member comprising:
a handle;
a hollow first shaft with a first end attached to the handle and an opposing second end, the first shaft having a channel with a proximal section having a first width and a distal section having a larger second width that tapers outward towards the second end;
a second shaft positioned in the channel;
a gripping member attached to the second shaft and positioned in the distal section of the channel and away from the handle, the gripping member including a base that extends across the distal section of the channel and a pair of arms extending outward from the base and being positioned on opposing sides of the distal section of the channel, each of the arms having a tip opposite from the base;
the gripping member including a tapered width that is narrower at the base and wider at the tips, the base further being narrower than the distal section of the channel and the tips being wider than the distal section of the channel;
the gripping member being movable along the distal section of the channel between a first position with the tips of the arms positioned a first distance apart and a second position with the tips in closer proximity to the handle and the tips of the arms positioned a smaller second distance apart.

9. The surgical instrument of claim 8, wherein outer surfaces of the arms contact against the distal section of the channel in the second position.

10. The surgical instrument of claim 8, wherein the second shaft is solid.

11. The surgical instrument of claim 8, further comprising a biasing member positioned between the arms of the gripping member, the biasing member applying an outward force to the arms to maintain the arms in contact with the sides of the channel.

12. The surgical instrument of claim 8, wherein the gripping member includes a U-shape with a closed end at the base and an open end between the arms.

13. The surgical instrument of claim 8, wherein a length of the distal section is greater than a length of the gripping member.

14. The surgical instrument of claim 8, wherein the distal section includes a non-tapered section with a width that is greater than the proximal section.

15. The surgical instrument of claim 8, further comprising ledges that extend inward from the inner surfaces of the first shaft to define the intersection of the distal section and the proximal section, the ledges being transverse to the centerline of the channel.

16. A method of grasping an elongated member using a surgical instrument during a surgical procedure, the method comprising:
positioning a pair of arms on a gripping member in an open orientation with distal sections of the arms spaced apart a first distance;
while in the open orientation, positioning the pair of arms on opposing sides of the elongated member;
moving proximal sections of the pair of arms into a tapered section of a tube and contacting outer surfaces of the arms against tapered ramps;
moving the proximal sections along the tapered ramps and forcing the arms closer together and compressing the distal sections of arms against the elongated member; and
placing the gripping member in a closed orientation secured to the elongated member and maintaining the arms in an enlarged distal section of the tapered section of the tube and in contact with the tapered ramps.

17. The method of claim 16, further comprising compressing a biasing member positioned between the arms while moving the proximal sections along the tapered ramps.

18. The method of claim 16, further comprising moving a shaft that extends outward from a proximal end of the pair of arms along the tube, the shaft extending between the pair of arms and a handle.

19. The method of claim 16, further comprising positioning the gripping member across the tube.

20. The method of claim 16, further comprising pulling the elongated member into contact with scalloped edges on the tube with the distal sections of the arms remaining outward beyond the tube.
